(19) United States
(12) Patent Application Publication
(10) Pub. No.: US 2023/0114594 A1
(43) Pub. Date: Apr. 13, 2023

(54) NEW THERAPEUTIC USE OF ANAKINRA FOR COVID-19

(71) Applicant: Swedish Orphan Biovitrum AB (publ), Stockholm (SE)

(72) Inventor: Cristina DE MIN, Plan-Les-Ouates Geneva (CH)

(73) Assignee: Swedish Orphan Biovitrum AB (publ), Stockholm (SE)

(21) Appl. No.: 17/906,199

(22) PCT Filed: Mar. 16, 2021
(86) PCT No.: PCT/EP2021/056685

§ 371 Date: Sep. 13, 2022

(30) Foreign Application Priority Data
Mar. 16, 2020 (EP) 20163457.3

(51) Int. Cl. A61K 38/20; A61K 31/573; A61K 45/06; A61P 31/14

(52) U.S. Cl. CPC A61K 38/2006; A61K 31/573; A61K 45/06; A61P 31/14

(57) ABSTRACT

A method treats a SARS-CoV-2 infection with anakinra. In particular the method treats or alleviates symptoms of respiratory distress and/or hyperinflammation in a patient. The treatment includes administering to a patient in need thereof a daily dose of at least about 400 mg of anakinra.

### FIELD OF THE INVENTION

The present invention relates to new uses of the recombinant human interleukin 1 receptor antagonist anakinra.

### BACKGROUND OF THE INVENTION

Severe acute respiratory syndrome coronavirus 2 (SARS-CoV-2) is the cause of the ongoing pandemic of coronavirus disease 2019 (COVID-19) that has been designated a Public Health Emergency of International Concern by the World Health Organization (WHO). Even though the infection is in most case manifested only by mild symptoms of disease, SARS-CoV-2 poses a great threat to many individuals, in particular elderly persons and individuals of previous or underlying health problems. Given the severity of the disease and the extremely high number of individuals affected, there is an urgent need for effective therapies.

It has been found that some COVID-19 patients develop a hyperinflammatory condition. A cytokine profile resembling secondary haemophagocytic lymphohistiocytosis (sHLH) is associated with disease severity, and is has been hypothesized that immunosuppression is likely to be beneficial (P Mehta, D F McAuley, M Brown, E Sanchez, R S Tattersall, J J Manson, Lancet, 395:10229, 13 Mar. 2020, p 1033-1034).

### SUMMARY OF THE INVENTION

It is an object of the present invention to provide new therapies for subjects suffering from COVID-19, that is, SARS-CoV-2 infection.

It is also an object of the present disclosure to provide a therapeutic agent which may be used alone or in combination with other agents for treatment of SARS-CoV-2 infection, in particular to treat or alleviate severe symptoms, such as hyperinflammation and/or respiratory distress, in subjects afflicted by SARS-CoV-2 infection.

According to a first aspect of the invention, this and other objects are achieved by anakinra for use in treatment of SARS-COV-2-infection in a subject, said treatment comprising administering to a said subject a daily dose of at least 400 mg of anakinra.

In another aspect, the invention provides methods of treatment of SARS-CoV-2-infection, and/or symptoms thereof, in subjects suffering from said infection, said treatment comprising administering to a said subject a daily dose of at least 400 mg of anakinra, or about 400 mg of anakinra.

These, and other objects which are evident to the skilled person from the present disclosure, are met by the different aspects of the invention as claimed in the appended claims and as generally disclosed herein.

It has been found that hyperinflammation, caused by a cytokine storm resulting from an exaggerated response of the immune system to the presence of the virus, represents one of the most important negative prognostic factor in patients infected with SARS-CoV-2. The term “cytokine storm syndromes” denote conditions characterized by fulminant hypercytokinaemia with high mortality. Cytokine storms can occur secondary to autoimmune or inflammatory disorders, infections or haematological malignancies.

The present invention was made in an attempt to address the current medical emergency, given the severity of the disease and the extremely high number of individuals affected. It is believed that the treatment regimen proposed herein may reduce the number of patients requiring mechanical ventilation. This is intended to address the most urgent need to preserve the access to intense care unit support to the lower possible number of patients and may potentially reduce mortality.

The interleukin-1 family is a group of proteins involved in regulating immune and inflammatory responses. Anakinra is a recombinant form (17 kDa polypeptide) of a naturally occurring human interleukin-1 receptor antagonist (IL-1ra) protein, functioning as a competitive inhibitor for receptor binding of IL-1. IL-1ra has a balancing function with regard to other pro-inflammatory variants of IL-1. Anakinra is approved for treatment of rheumatoid arthritis, systemic juvenile idiopathic arthritis (sJIA) and adult-onset Still's disease and cryopurin-associated periodic syndromes, at a dose of 100 mg once daily, or in weight-based doses up to 8 mg/kg/day, by subcutaneous (s.c.) injection.

However, anakinra has also been administered off-label in higher doses in critically ill patients suffering from macrophage activation syndrome (MAS)/sHLH and septic chock, including daily doses of up to 400 mg/day, with successful outcome (Grom A A, Horne A, De Benedetti F. Macrophage activation in the era of biologic therapy. Nat Rev Rheumatol 2016; 12:259-68; Shakoory B, Carcillo J A, Chatham W W et al. Interleukin-1 receptor blockade is associated with reduced mortality in sepsis patients with features of macrophage activation syndrome: reanalysis of a prior phase III trial. Crit Care Med 2016; 44:275-81; Kahn P J, Cron R Q. Higher-dose anakinra is effective in a case of medically refractory macrophage activation syndrome. J Rheumatol 2013; 40:5).

MAS and septic chock are acute hyperinflammatory conditions that share some molecular features with hyperinflammation in COVID-19, such as release of various interleukins (including IL-1) and interferon-y.

Intravenous (i.v.) administration of anakinra has been studied in clinical trials in healthy volunteers and in critically ill patients with sepsis and hyper-inflammation at variable i.v. doses up to 3500 mg/dag over 72 hours: e.g. 2 mg/kg/hour, 20 mg/kg/day (<40 kg) and 916 mg/day (>40 kg), bolus of 100 mg followed by infusion of 2 mg/kg/hour, without safety concerns.

The present inventor has identified that a daily dose of anakinra of 400 mg may be useful in the treatment of severe effects of SARS-COV-2 infection, also referred to as COVID-19. In particular, four administrations per day, each of 100 mg of anakinra, is considered promising.

Hence, in embodiments, said daily dose of 400 mg of anakinra is distributed over four administration occasions of 100 mg each. In embodiments, said administration occasions are from about 4 to about 8 hours apart, such as about 6 hours apart.

To clarify, the daily dose of 400 mg of anakinra is the total dose administered during one day, in other words the administration of 400 mg anakinra is distributed over the course of one day.

Hence, as used herein, the term “daily dose” refers to the total dose administered during one day to a patient.

As used herein, the term “day” is to be interpreted as a continuous 24-hour period.

As used herein, the term “administration occasion” refers to a limited time period wherein a drug is administered to a patient in need thereof. To clarify, the administration occasion encompasses the time period which is required for the administration of the entire dose of the drug which is to be administered at this time/occasion. For example, an administration occasion may be the time period required for one or several injections (e.g. intravenous infusion), said one or several injections representing the complete dose to be administered at the specific time/occasion. Also, an administration of a dose which requires for example a 3-hour intravenous administration is to be regarded as one administration occasion. It is to be understood that the time period of an administration occasion is significantly shorter than the period in between two administration occasions. For example, if a dose of a drug to be administered by subcutaneous injection is 100 mg, the administration of two injections of 50 mg is considered to be encompassed by the same administration occasion, provided that the time period between the administration of said two 50 mg injections is shorter than the intended period between a first administration of an entire dose of 100 mg and a second administration of an entire dose of 100 mg.

The treatment with anakinra may be carried out or continued for at least 7 days, such as at least 10 days, such as at least 14 days, such as at least 21 days. For example, the treatment may be carried out or continued for 14 days, or 15 days. In embodiments, the daily dose may be administered on day 1 to day 14, or on day 1 to day 15, of a treatment period.

For administration of high-dose anakinra, intravenous administration may be preferred over subcutaneous injection. The terminal half-life of anakinra when delivered subcutaneously is longer than when delivered intravenously, likely due to slower absorption. Also, the maximum plasma concentration (Cₘₐₓ) of anakinra is higher, and the time to reach it (Tₘₐₓ) is faster, with intravenous dosing compared to subcutaneous dosing (Yang B B, Gozzi P, Sullivan J T. Clin Transl Sci 2019; 12:371-78, Yang B B, Baughman S, Sullivan J T. Clin Pharmacol Ther 2003; 74:85-94).The short half-life of a drug may be considered an advantage when used in treatment of critically ill patients since it allows faster clearance of the drug following discontinuation.

Anakinra may be administered by intravenous (i.v.) infusion or subcutaneous injection, and preferably by i.v. infusion, according to embodiments of the present invention. In view of the short half-life, and the faster and higher maximum plasma concentration that may be obtained with intravenous dosing, it may be particularly preferable to distribute the daily dosing over several administration occasions, such as four i.v. administrations of 100 mg each. The administration occasions may be evenly distributed over a day (24 hours). Hence, in embodiments, the treatment may comprise i.v. administration of 100 mg of anakinra about every 6 hours, thus making a total daily dose of 400 mg.

In embodiments, the treatment may comprise administration of a further therapeutic agent. The further therapeutic agent may be selected from the group consisting of: anti-inflammatory agents, antibiotics, anti-fungal agents, anti-viral agents. In embodiments, the anti-inflammatory agent is methylprednisolone.

Methylprednisolone may be administered at a daily dose of at least 15 mg, such as at least 30 mg, such as at least 60 mg. The treatment may involve administration of a daily dose of 30 mg or 60 mg of methylprednisolone for at least 3 days, such as at least 5 days.

In embodiments, a daily dose of methylprednisolone may be distributed over three administration occasions. For instance, methylprednisolone may be administered at a daily dose of 60 mg, distributed over three administration occasions, on day 1 to day 5 of a treatment period. Further, in embodiments, the treatment may comprise administration of methylprednisolone at a daily dose of 30 mg, distributed over three administration occasions, on day 6 to day 10 of a treatment period. Further, such treatment may comprise administration of methylprednisolone at a daily dose of 15 mg, distributed over three administration occasions, on day 11 to day 14 of a treatment period.

The treatment of SARS-CoV-2 infection may be treatment of at least one of hyperinflammation and respiratory distress. Hence, in embodiment, the subject to be treated may exhibit signs of respiratory distress and/or hyperinflammation.

In the present context, the following are considered to be signs of respiratory distress in a subject: PaO2/FiO2 ≤300 mmHg and >200 mmHg, RR ≥30 breaths/min, SpO2 ≤93% in air at rest.

In the present context, the following are considered to be signs of hyperinflammation in a subject: Ferritin >500 ng/mL, LDH >300 U/L, D-dimers >1000 ng/m L.

In embodiments, the treatment does not include concomitant administration of one or more of the following agents: tocilizumab, canakinumab, TNF inhibitors, JAK inhibitors, hydroxychloroquine.

The subject may be a human subject.

### DETAILED DESCRIPTION

SARS-CoV-2 belongs to the broad family of viruses known as coronaviruses. It is a positive-sense single-stranded RNA (+ssRNA) virus. Its RNA sequence is approximately 30,000 bases in length. The SARS-CoV-2 genome sequence information has been made publicly available and can be found e.g. in the NCBI database, genome ID: MN908947.

Components in a protein drug formulation of anakinra may include buffering agents, tonicity agents, antioxidants, stabilizers, surfactants, bulking agents, chelating agents and preservatives. Kineret®, a formulation of anakinra at pH 6.5 suitable for injection, contains sodium citrate, sodium chloride, disodium EDTA, polysorbate 80 and water.

The following clinical study protocol synopsis describes a phase 2/3, randomized, open-label, parallel group, 3-arm, multicenter study to investigate the efficacy and safety of intravenous administrations of anakinra, an interleukin-1 (IL-1) receptor antagonist, and emapalumab, an anti-interferon gamma (anti-IFNγ) monoclonal antibody, versus standard of care in reducing hyper-inflammation and respiratory distress in patients with SARS-CoV-2 infection.

Main Study Objectives

The main objectives of the study are:
To assess the effect of emapalumab and anakinra on hyper-inflammation and pulmonary function in patients with SARS-CoV-2 infection
To evaluate the safety and tolerability profile of intravenous (i.v.) administrations of emapalumab and anakinra in patients with SARS-CoV-2 infection
To confirm the proposed dosing regimen of emapalumab and anakinra.

Study Population

Patients with documented SARS-CoV-2 infection with respiratory distress and hyperinflammation.

Inclusion Criteria
Patients of both genders
Age >30 years and <80 years
Documented presence of SARS-CoV-2 infection
Presence of respiratory distress defined as:
   PaO2/FiO2 <300 mm Hg and >200 mm Hg or
   RR ≥30 breaths/min or
   SpO2 ≤93% in air at rest
Note: Patients in CPAP are eligible.
Presence of hyper-inflammation defined as:
   Ferritin >500 ng/mL
   LDH >300 U/L
   D-dimers >1000 ng/mL
Signed informed consent provided by the patient, or by the patient's legally authorized representative(s), as applicable.

Exclusion Criteria
Patients in mechanical ventilation or with MEWS score >4 with evidence of moderate or above ARDS (Berlin definition, namely with PaO2/FiO2 >100, but ≤200 mm Hg) or severe respiratory insufficiency or evidence of rapid worsening (respiratory distress requiring mechanical ventilation or presence of shock or presence of concomitant organ failure requiring ICU admission)
Impairment of cardiac function defined as poorly controlled heart diseases, such as NYHA class II (mild*) and above, cardiac insufficiency, unstable angina pectoris, myocardial infarction within 1 year before enrollment, supraventricular or respiratory insufficiency or evidence of rapid worsening (respiratory distress requiring mechanical ventilation or presence of shock or presence of concomitant organ failure requiring ICU admission)
Impairment of cardiac function defined as poorly controlled heart diseases, such as NYHA class II (mild*) and above, cardiac insufficiency, unstable angina pectoris, myocardial infarction within 1 year before enrollment, supraventricular or ventricular arrhythmia need treatment or intervention.
* Patients with cardiac disease resulting in slight limitation of physical activity. They are comfortable at rest. Ordinary physical activity results in fatigue, palpitation, dyspnea, or anginal pain
Severe renal dysfunction (estimated glomerular filtration rate ≤30 mL/min/1.73 m²) or receive continuous renal replacement therapy, hemodialysis, or peritoneal dialysis
Uncontrolled hypertension (seated systolic blood pressure >180 mmHg, or diastolic blood pressure >110 mmHg)
Administration of plasma from patients recovered from SARS-CoV-2 infection
Clinical suspicion of latent tuberculosis
History of hypersensitivity or allergy to any component of the study drug
Pregnancy

Study Drug

Anakinra is the recombinant form of the IL-1 human receptor antagonist (IL-1ra). Emapalumab is a fully human IgG1 monoclonal antibody (mAb) neutralizing human IFNγ.

Dosing Regimen, Frequency of Administration and Treatment Duration:

Anakinra will be administered at a total dose of 400 mg per day, divided in 4 i.v. doses of 100 mg every 6 hours. Anakinra treatment will continue for 15 days i.e., days 1 to 15.

Emapalumab will be administered at the initial dose of 6 mg/kg by i.v. infusion i.e., Day 1. Emapalumab treatment will be continued at the dose of 3 mg/kg, every 3 days for a total of 4 additional infusions i.e., Days 4, 7, 10 and 13.

Background Therapy and Concomitant Medication

All patients participating in the study will receive background therapy with methylprednisolone. methylprednisolone will be given at 20 mg×3/day for 5 days, then 10 mg×3/day for 5 days, then 5 mg×3/day for 4 days. Concomitant use of tocilizumab, canakinumab, TNF inhibitors, JAK inhibitors and hydroxychloroquine is not allowed.

Antimicrobial therapy and prophylaxis are not limited.

Analgesic treatment, transfusion of blood products, electrolyte and glucose infusions, i.v. parenteral nutrition, inotropic support, antibiotics, anti-fungal and anti-viral treatments, ultrafiltration or hemodialysis, as well as general supportive care are permitted.

Sample Size

54 patients in total

Study Duration and Study End Definition

The study consists of screening, a 2-week treatment period and an 8-week follow-up period. The 2-week treatment period is open, and the patients will be randomized to treatment with emapalumab, anakinra or standard of care in a 1:1:1 ratio. The primary endpoint will be evaluated at Day 15.

A follow-up by visit or phone call will be performed 4 and 8 weeks after the end of the treatment period (Weeks 6 and 10).

The study duration for an individual patient will not exceed 10 weeks.

The end of the study is defined as last patient last follow-up visit/phone call (LPLV).

Data Review Committee

A committee composed of experts in intensive care, inflammation, infectious diseases will be involved in study oversight and interpretation of the study results.

Study Endpoints

The assessment of emapalumab and anakinra efficacy in this patient population will be based on:
Primary endpoint: Rate of treatment success, defined as the percentage of patients not requiring any of the followings:
   Invasive mechanical ventilation or
   Extracorporeal membrane oxygenation (ECMO) or
   Continuous positive airway pressure (CPAP)
Secondary efficacy endpoints:
   Time to mechanical ventilation
   Change from baseline in MEWs score
   Evolution of hyperinflammatory parameters during treatment until SD15 with measurements performed every 3 days: Ferritin, LDH, D-dimers.
   Evolution of other relevant laboratory parameters during treatment until SD15 with measurements performed every 3 days: WBC with differential counts, RBC, Hb, Platelet count, Fibrinogen, Complement C3/C4, Prothrombin Time, Cardiac troponin, Liver tests (AST, ALT, total bilirubin levels), CRP and ESR, Creatinine, Survival
Safety Endpoints:
   Treatment-emergent severe fatal and life-threatening serious adverse events (SAEs).
   Adverse events leading to premature discontinuation of study treatment.
   Anaphylactic/anaphylactoid reactions
   Emapalumab treatment emergent AEs of special interest: infections caused by pathogens potentially favored by IFN-y neutralizations such as mycobacteria, salmonella, shigella, herpes zoster, and histoplasma capsulatum; severe infusion related reactions
   Anakinra treatment emergent AEs of special interest: severe neutropenia
   Treatment-emergent laboratory abnormalities.
CXCL9, IL-1, IL-6, sIL-2R and selected exploratory parameters every 3 days, whenever possible.

Visits Schedule and Assessments

The clinical and laboratory parameters to be collected at given time points are indicated in Table 1 (Schedule of Events). This schedule also includes information on the patient's demographics, medical history and prior medication. High resolution CT scan is required at screening (within 72 hours prior to start of treatment) to document the presence of pulmonitis. All parameters indicated in the Schedule of Events will be assessed at the study site.

Statistics

Each of the study drug treatment arms is compared independently to standard of care.

The trial design consists of two stages, with equal numbers of patients randomised into Stage 1 and into Stage 2 per treatment arm.

At the end of Stage 1 the success rates are compared between each of the two treatment arms and standard of care. There is the potential to stop for futility or for efficacy of emapalumab or anakinra. Note: these rules are binding.

If the trial continues beyond Stage 1, the groups will be compared to standard of care at the end of Stage 2 for efficacy. The overall one-sided significance level for efficacy for either emapalumab or anakinra is 0.097 (9.7%) to allow the initial acceptance of a potentially valuable new treatment.

The design has a total sample size of 54, 27 recruited in Stage 1 and 27 recruited in Stage 2 with equal 1:1:1 randomisation.

This design has the following characteristics for the comparison of each of emapalumab and anakinra with standard of care:
Stop either emapalumab or anakinra (or both) at the end of Stage 1 for futility if the one-sided p-value in favour of emapalumab/anakinra is >0.690
Stop either emapalumab or anakinra (or both) at the end of Stage 1 for demonstration of efficacy if the one-sided p-value in favour of emapalumab or anakinra is <0.025
If the trial continues for either emapalumab or anakinra (or both) to

Stage 2, declare efficacy at the end of Stage 2 if the one-sided p-value in favour of emapalumab or anakinra is <0.159.

This design has acceptable properties in terms of the false positive potential, controlling the overall type I error at 7.9% for each of the 2 comparisons. The power of this design for each of those comparisons is 74% under the assumption that the true success rates are 50% in the SoC group increasing to 80% in the emapalumab or anakinra groups. The calculations on the operating characteristics of this design have been undertaken using PASS, Version 14: Group-Sequential Tests for Two Proportions (Simulation).

The value for the type I error has been chosen in recognition of the urgent unmet medical need to allow the identification of a signal, at least, from a statistical perspective. Having seen a statistical signal, it is then a matter of evaluating whether the observed treatment differences represent clinically relevant effects that can satisfy that unmet need.

In case indications of efficacy require to be confirmed, additional patients may be added into this study. In case the outcome is statistically convincing, i.e. statistically significant on a 5% level in the stage 2 analysis, efficacy will be considered confirmed and the results will, where warranted, be used to seek regulatory approvals.

TABLE 1
Schedule of events

| | SCREENING | TREATMENT PERIOD | | | | | | | |
|---|---|---|---|---|---|---|---|---|---|
| | Up to 3 | Visit 1 | | | | | | FOLLOW-UP PERIOD | |
| | days prior | Day 1 | Visit 2 | Visit 3 | Visit 4 | Visit 5 | Visit 6 | Visit 7/TC | Visit 8/TC |
| ASSESSMENT | to Visit 1 | (Baseline) | Day 4 | Day 7 | Day 10 | Day 13 | Day 15 | Week 6 | Week 10 |
| Informed consent | X | | | | | | | | |
| Eligibility criteria | X | X¹ | | | | | | | |
| Patient information² | X | | | | | | | | |
| Physical examination | X | | | | | | | | |
| Vital signs assessment³ | X | X | X | X | X | X | X | | |
| ECG assessment | X | | | | | | X | | |
| CT scan | X⁴ | | | | | | X | | |
| Laboratory assessments (local)⁵ | X | X | X | X | X | X | X | | |
| Randomization | | X | | | | | | | |
| IMP administration | | X-----------------------------------------------------------X | | | | | | | |
| Concomitant medication | | X | X | X | X | X | X | | |
| incl background therapy⁶ | | | | | | | | | |
| Assessment of pulmonary | | X | X | X | X | X | X | | |
| function⁷ | | | | | | | | | |
| Survival | | | X | X | X | X | X | X | X |
| Hospital discharge | | | | | | | | X | X |
| Adverse events⁸ | | X | X | X | X | X | X | X | X |
| Biomarkers (CXCL9, IL-1, | | X | X | X | X | X | X | | |
| IL-6, sIL-2R)⁹ | | | | | | | | | |

Abbreviations: CPAP, continuous positive airway pressure; CT, computed tomography; ECG, electrocardiogram; ECMO, extracorporeal membrane oxygenation; FDP, Fibrinogen Degradation Products; IMP, Investigational Medicinal Product
During the treatment period (Days 1 to 14), clinical and laboratory assessments and procedures should preferably be performed before IMP administration.
¹Before IMP administration.
²Includes demographics, medical history and prior medication.
³Body temperature, blood pressure, heart rate, respiratory rate and oxygen saturation.
⁴Not to be repeated if performed within 72 hours of the first IMP administration.
⁵Ferritin, LDH, D-dimers. WBC with differential counts, RBC, Hb, Platelet count, Fibrinogen, Complement C3/C4, Prothrombin Time, Cardiac troponin, AST, ALT, total bilirubin levels, CRP, ESR and Creatinine.
⁶For details, see section on background and concomitant medication.
⁷Requirement for invasive mechanical ventilation, ECMO, CPAP or high flow oxygen devices and MEWs score.
⁸Incidence, severity, causality and outcomes of treatment-emergent severe fatal and life-threatening serious AEs, including anaphylactic and anaphylactoid reactions to study drugs, adverse events leading to premature discontinuation of study treatment and adverse events of special interest.
⁹If possible

The person skilled in the art realizes that the present invention by no means is limited to the preferred embodiments described above. On the contrary, many modifications and variations are possible within the scope of the appended claims.

Additionally, variations to the disclosed embodiments can be understood and effected by the skilled person in practicing the claimed invention, from a study of the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1-34. (canceled)

35. A method of treatment of a SARS-COV-2-infection in a subject in need thereof, said treatment comprising:
administering a daily dose of at least about 400 mg of anakinra to the subject in need thereof.

36. The method of treatment of a SARS-COV-2-infection according to claim 35, wherein said daily dose is distributed over four administration occasions of 100 mg each.

37. The method of treatment of a SARS-COV-2-infection according to claim 36, wherein said administration occasions are from about 4 to about 8 hours apart.

38. The method of treatment of a SARS-COV-2-infection according to claim 37, wherein said administration occasions are about 6 hours apart.

39. The method of treatment of a SARS-COV-2-infection according to claim 35, wherein said treatment is continued for at least 7 days.

40. The method of treatment of a SARS-COV-2-infection according to claim 35, wherein anakinra is administered by intravenous (i.v.) infusion.

41. The method of treatment of a SARS-COV-2-infection according to claim 35, wherein said treatment further comprises administration of a further therapeutic agent selected from the group consisting of one or more of: an anti-inflammatory agent, an antibiotic, an anti-fungal agent, and an anti-viral agent.

42. The method of treatment of a SARS-COV-2-infection according to claim 41, wherein said agent is an antiinflammatory agent.

43. The method of treatment of a SARS-COV-2-infection according to claim 42, wherein said anti-inflammatory agent is methylprednisolone.

44. The method of treatment of a SARS-COV-2-infection according to claim 43, wherein methylprednisolone is administered at daily dose of at least 15 mg.

45. The method of treatment of a SARS-COV-2-infection according to claim 44, wherein said daily dose of methylprednisolone is distributed over three administration occasions.

46. The method of treatment of a SARS-COV-2-infection according to claim 44, wherein methylprednisolone is administered at a daily dose of 60 mg for at least 3 days.

47. The method of treatment of a SARS-COV-2-infection according to claim 44, wherein methylprednisolone is administered at a daily dose of 60 mg, distributed over three administration occasions, on day 1 to day 5 of a treatment period.

48. The method of treatment of a SARS-COV-2-infection according to claim 44, wherein methylprednisolone is administered at a daily dose of 30 mg distributed over three administration occasions for at least 3 days.

49. The method of treatment of a SARS-COV-2-infection according to claim 48, comprising administration of methylprednisolone at a daily dose of 30 mg, distributed over three administration occasions, on day 6 to day 10 of a treatment period.

50. The method of treatment of a SARS-COV-2-infection according to claim 44, wherein methylprednisolone is administered at a daily dose of 15 mg distributed over three administration occasions for at least 4 days.

51. The method of treatment of a SARS-COV-2-infection according to claim 50, wherein methylprednisolone is administered at a daily dose of 15 mg, distributed over three administration occasions, on day 11 to day 14 of a treatment period.

52. The method of treatment of a SARS-COV-2-infection according to claim 35, wherein said treatment does not include concomitant administration of one or more of the following agents: tocilizumab, canakinumab, TNF inhibitors, MK inhibitors, and/or hydroxychloroquine.

53. The method of treatment of a SARS-COV-2-infection according to claim 35, wherein the treatment comprises a treatment of symptoms of SARS-CoV-2 infection.

54. The method of treatment of a SARS-COV-2-infection according to claim 53, wherein said treatment of symptoms is for the symptoms of hyperinflammation and/or respiratory distress.

55. The method of treatment of a SARS-COV-2-infection according to claim 54, wherein said subject exhibits signs of respiratory distress.

56. The method of treatment of a SARS-COV-2-infection according to claim 55, wherein said subject exhibits one or more of the following signs of respiratory distress: PaO2/FiO2 ≤300 mmHg and >200 mmHg, RR ≥30 breaths/min, and/or SpO23 93% in air at rest.

57. The method of treatment of a SARS-COV-2-infection according to claim 54, wherein said subject exhibits signs of hyperinflammation.

58. The method of treatment of a SARS-COV-2-infection according to claim 57, wherein said subject exhibits one or more of the following signs of hyperinflammation: Ferritin >500 ng/mL, LDH >300 LA, and/or D-dimers >1000 ng/mL.
